(19) United States
(12) Patent Application Publication
(10) Pub. No.: US 2017/0086670 A1
(43) Pub. Date: Mar. 30, 2017

(54) APPARATUS FOR MONITORING AN EYE AND/OR ORBITAL REGION DURING SURGERY, AND ASSOCIATED METHOD

(71) Applicant: TIDAL KEY GROUP, LLC, London, TN (US)

(72) Inventor: MATTHEW DAVID LOWRANCE, LENOIR CITY, TN (US)

(21) Appl. No.: 14/871,204

(22) Filed: Sep. 30, 2015

(51) Int. Cl. A61B 3/16; A61B 5/00; A61B 5/0496

(52) U.S. Cl. CPC A61B 3/16; A61B 5/0496; A61B 5/6821; A61B 5/6832; A61B 5/746; A61B 5/0048; A61B 2505/05; A61B 2560/0468; A61B 2562/0247; A61B 2562/125

(57) ABSTRACT

An apparatus is provided for monitoring an orbital area, including an eye, of a patient during surgery. A flexible pressure-sensitive element is configured to be responsive to a change in pressure applied thereto. The pressure-sensitive element is adapted to be applied over and adhered to the orbital area so as to be disposed over and conformed to a closed eyelid and the eye associated therewith, to sense pressure affecting the eye of the patient. A measuring arrangement is in communication with the pressure-sensing element, wherein the measuring arrangement is configured to determine a change in a property of the pressure-sensitive element in response to the pressure-sensitive element experiencing a change in pressure applied thereto. Associated methods are also provided.

### BACKGROUND

Field of the Disclosure

The present disclosure generally related to surgical apparatuses and associated methods and, more particularly, to an apparatus and associated method for monitoring an orbital region, including an eye, of a patient during surgery.

Description of Related Art

Post-operative vision loss is a relatively rare but devastating condition for a surgical patient for which there are no effective treatment options. Methods and apparatuses for detailed monitoring of the cardiovascular, respiratory, and nervous systems of the patient during surgery are well developed and routinely used to track the physiologic condition of anesthetized patients, before, during, and after the surgical procedure. However, no effective method or apparatus exists for monitoring visual function of the anesthetized patient, despite the fact that the window for successful countermeasure is closed by the time the injury is recognized post-operatively.

Some existing products are designed to prevent post-operative vision loss (POVL) by protecting the eye and orbit from inadvertent external pressure during prone surgery. However, such devices are often a modification of protective goggles, and may be the cause of POVL in some cases. In other instances, manual eye checks may be performed periodically during the surgery, sometimes assisted by mirrors positioned for facilitating viewing of the eyes of the patient in the prone position. However, such a solution requires time, effort, and active and periodic monitoring by a clinician who is otherwise occupied with the surgical procedure.

Post-operative recognition or determination of vision loss, following any opportunity for remediation, often results in permanent vision loss or impairment and carries an associated catastrophic impact on the patient's quality of life. As such, there exists a need for an apparatus, and method for monitoring an orbital region, including an eye, of a patient during surgery so as to allow a medical team to take remedial action is response to any adverse conditions affecting the eye, while the opportunity exists, so as to preserve the patient's vision.

### SUMMARY OF THE DISCLOSURE

The above and other needs are met by aspects of the present disclosure which, in one aspect, provides an apparatus for monitoring an orbital area, including an eye, of a patient during surgery. Such an apparatus comprises a flexible pressure-sensitive element configured to be responsive to a change in pressure applied thereto, wherein the pressure-sensitive element is adapted to be applied over and adhered to the orbital area so as to be disposed over and conformed to a closed eyelid and the eye associated therewith, to sense pressure affecting the eye of the patient. A measuring arrangement is in communication with the pressure-sensing element, wherein the measuring arrangement is configured to measure a change in a property of the pressure-sensitive element in response to the pressure-sensitive element experiencing a change in pressure applied thereto.

Another aspect of the disclosure provides a method of forming an apparatus for monitoring an orbital area, including an eye, of a patient during surgery. Such a method comprises engaging a flexible pressure-sensitive element with a measuring arrangement, wherein the pressure-sensitive element is configured to be responsive to a change in pressure applied thereto and is adapted to be applied over and adhered to the orbital area so as to be disposed over and conformed to a closed eyelid and the eye associated therewith, to thereby sense pressure affecting the eye of the patient, and wherein the measuring arrangement is configured to determine a change in a property of the pressure-sensitive element in response to the pressure-sensitive element experiencing a change in pressure applied thereto.

Yet another aspect of the disclosure provides a method of monitoring an orbital area, including an eye, of a patient during surgery. Such a method comprises applying and adhering a flexible pressure-sensitive element, configured to be responsive to a change in pressure applied thereto, over and to the orbital area such that the pressure-sensitive element is disposed over and conformed to a closed eyelid and the eye associated therewith, to sense pressure affecting the eye of the patient. Using a measuring arrangement in communication with the pressure-sensing element, a change in a property of the pressure-sensitive element is then determined in response to the pressure-sensitive element experiencing a change in pressure applied thereto.

Further features and advantages of the present disclosure are set forth in more detail in the following description.

### DESCRIPTION OF THE DRAWINGS

Having thus described the disclosure in general terms, reference will now be made to the accompanying drawings, which are not necessarily drawn to scale, and wherein:

FIG. 1 schematically illustrates an apparatus for monitoring an orbital region, including an eye, of a patient during surgery, according to one aspect of the disclosure;

FIGS. 2 and 3 schematically illustrate an apparatus for monitoring an orbital region, including an eye, of a patient during surgery, according to the aspect of the disclosure as shown in FIG. 1, applied to the orbital region of a human head;

FIGS. 4 and 5 schematically illustrate electrodes implemented by an apparatus for monitoring an orbital region, including an eye, of a patient during surgery, according to various aspects of the present disclosure;

FIGS. 6A and 6B schematically illustrate a determination of pressure applied to an orbital area of a patient in relation to a baseline, using an apparatus for monitoring an orbital region, including an eye, of a patient during surgery, according to one aspect of the present disclosure;

FIG. 7 schematically illustrates an electrophysiological device interfaced with an apparatus for monitoring an orbital region, including an eye, of a patient during surgery, according to one aspect of the present disclosure;

FIG. 8 schematically illustrates a method of forming an apparatus for monitoring an orbital area, including an eye, of a patient during surgery, according to one aspect of the present disclosure; and

FIG. 9 schematically illustrates a method of monitoring an orbital area, including an eye, of a patient during surgery, according to one aspect of the present disclosure.

### DETAILED DESCRIPTION OF THE DISCLOSURE

The present disclosure now will be described more fully hereinafter with reference to the accompanying drawings, in which some, but not all aspects of the disclosure are shown. Indeed, the disclosure may be embodied in many different forms and should not be construed as limited to the aspects set forth herein; rather, these aspects are provided so that this disclosure will satisfy applicable legal requirements. Like numbers refer to like elements throughout.

Certain terminology is used herein for convenience only and is not to be taken as a limitation on the scope of the disclosure. For example, words such as “upper,” “lower,” “left,” “right,” “horizontal,” “vertical,” “upward,” and “downward” merely describe the configuration shown in the figures. Indeed, the components may be oriented in any direction and the terminology, therefore, should be understood as encompassing such variations unless specified otherwise.

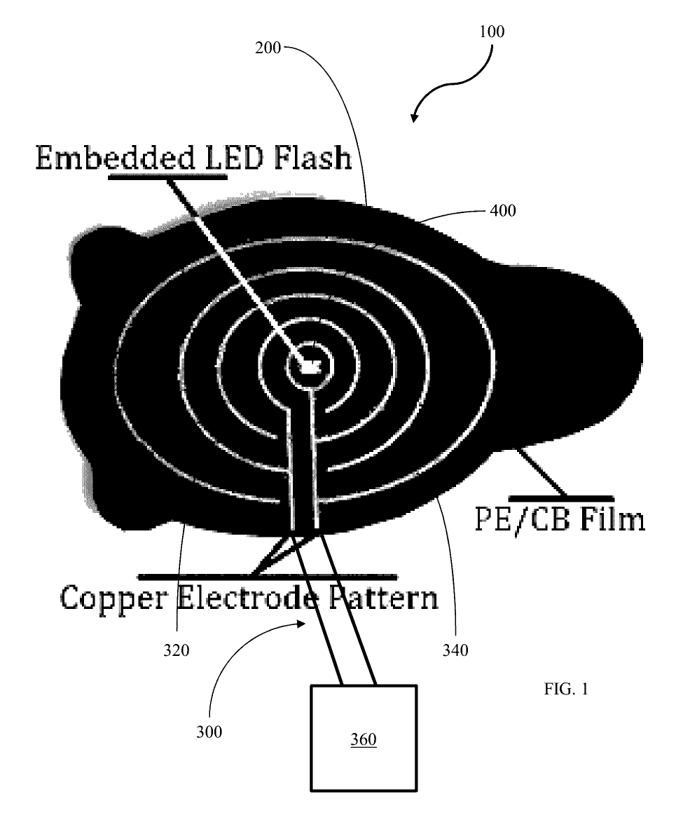

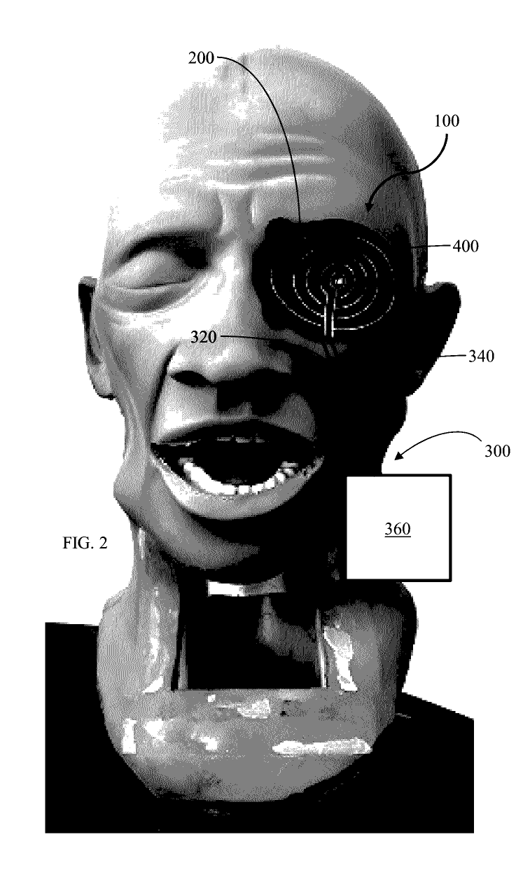

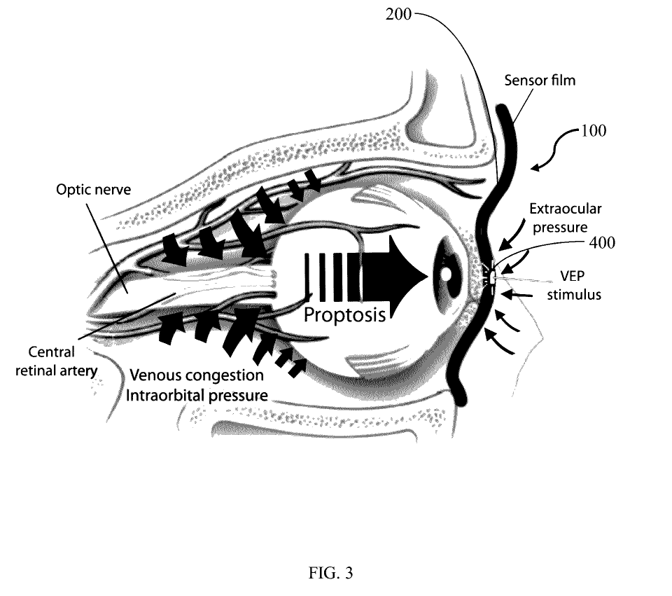

Aspects of the disclosure implement an apparatus for monitoring an orbital region, including an eye, of a patient during surgery, wherein such monitoring may provide early indicia of potentially adverse conditions affecting the eye so as to allow the surgical team to implement timely remedial action to minimize or eliminate the risk of post-operative vision loss. That is, aspects of the present disclosure are configured and intended to safeguard against, minimize, or eliminate post-operative visual changes or loss as a result of procedures performed under general anesthesia, particularly where the patient is placed in a prone position during the surgery (i.e., spinal surgery procedures). An exemplary apparatus according to the disclosure is schematically illustrated in FIGS. 1-3, and indicated generally as element 100. In one instance, the monitoring apparatus 100 includes a flexible pressure-sensitive element 200 configured, for example, to be responsive to a change in pressure applied thereto, a deflection thereof, or a strain experienced thereby. As shown in FIGS. 2 and 3, the pressure-sensitive element 200 is sized so as to at least cover the orbital area of a human head. When applied to the orbital area, the pressure-sensitive element 200 is disposed over the eye. To prevent direct external contact with the eye, and to prevent the eye from drying out, it may be preferable for the pressure-sensitive element 200 to be applied over a closed eyelid and to hold the eyelid closed. In order to maintain the eyelid in the closed position, the pressure-sensitive element 200 may have a temporary adhesive material engaged therewith in a manner, for example, similar to an adhesive bandage. Further, in order to closely monitor the condition of the eye itself (i.e., a pressure affecting the eye whether from an internal source or an external source), it may be preferable for the pressure-sensitive element 200 to be sufficiently flexible to conform to the orbital area, including the closed eyelid and the underlying eye.

In summary, the pressure-sensitive element 200 may a thin flexible and/or conformal film configured to be affixed over and adhered to the eye/orbital area during surgery, wherein the film may cover any portion of the ocular area, preferably at least the ocular area, but up to and including the closed eyelid and periorbital region beyond the circumference of the orbital area/region. In some instances, the pressure-sensitive element 200 may have a temporary adhesive material engaged therewith, wherein such a temporary adhesive material may be suitable for “stick-to-skin” medical applications. The temporary adhesive material is thus configured to cooperate with the pressure-sensitive element 200 to keep the eyelid in the closed position during the surgical procedure. In some instances, the pressure-sensitive element 200 may be engaged with and carried by a substrate (not shown) having a temporary adhesive material engaged therewith (i.e., similar to an adhesive bandage). Accordingly, in addition to or instead of the pressure-sensitive element 200 being directly adhered to the orbital region, the pressure-sensitive element 200 may be engaged with and carried by the substrate, wherein the substrate is then adhered to the orbital region via the temporary adhesive material engaged therewith.

In some aspects, the pressure-sensitive element 200 may itself, or in combination with the substrate, physically protect the eye and orbital area from injury during the surgical procedure, for example, by reducing the risk of or preventing skin abrasions, corneal abrasions, or the entrance of a foreign body into the eye. Moreover, the pressure-sensitive element 200 may be configured so as to be capable of sensing or otherwise indicating pressure on the eye/orbital area or region resulting from external contact with the eye/orbital area, and/or sensing or otherwise indicating pressure on and/or deflection of the pressure-sensitive element 200 due to changes in the geometry of the eye/orbital area, for example, due to increased venous congestion, changes in intracranial pressure, changes in intraorbital compartment pressure, changes in intraocular pressure, and/or voluntary or involuntary eye or eyelid movement.

In some aspects, the pressure-sensitive element 200 may comprise a nonconductive matrix having conductive elements distributed therein. In such instances, the matrix having the conductive elements dispersed therein may be configured to change resistivity or conductivity in response to deflection thereof, pressure applied thereto, or strain experienced thereby. In one example, the matrix may be comprised of a polymeric material, such as polyethylene. In another example, the conductive elements may be comprised of carbon black, carbon nanotubes, graphite, a metal, or a metal oxide. Pressure-sensitive materials or films of the type described herein may, in some instances be commercially available, for instance, under the trade name Velostat™, for general uses such as, for example, for dissipation of static electricity or as a pressure transducer. One skilled in the art will appreciate, however, that the pressure-sensitive element 200 may take many different forms. For example, in some instances, a capacitive-type pressure sensor may be implemented in a similar manner to the types of pressure-sensitive elements 200 disclosed herein.

With pressure-sensitive elements 200 of the types disclosed herein, the resistivity or conductivity of the may change in relation to a change in dimension of the film, wherein a change in dimension of the film may result from a pressure applied thereto, a deflection or bending thereof, or a strain experienced thereby. In order to sense or determine such a change in resistivity or conductivity, an appropriate measuring arrangement 300 may be disposed in communication with the pressure-sensing element 200. The measuring arrangement 300 may include a measuring device 360 configured to determine a change in a property of the pressure-sensitive element 200 in response to the pressure-sensitive element experiencing a change in pressure applied thereto, a change in deflection thereof, or a change in strain experienced thereby. As such, in some aspects, electrodes 320, 340 may be applied to the pressure-sensitive element 200 (see, e.g., FIGS. 1 and 2), wherein the electrodes 320, 340 may be spaced apart from each other by a selected dimension of the pressure-sensitive element 200. The electrodes 320, 340 may also extend and be spaced about the pressure-sensitive element 200 so as to be capable of sensing a change of the property over a greater extent of the pressure-sensitive element 200. For example, since the pressure-sensitive element 200 may extend over and across the orbital region of the human head, the electrodes 320, 340 may be applied to and extend about the pressure-sensitive element 200 so as to also cover the orbital region.

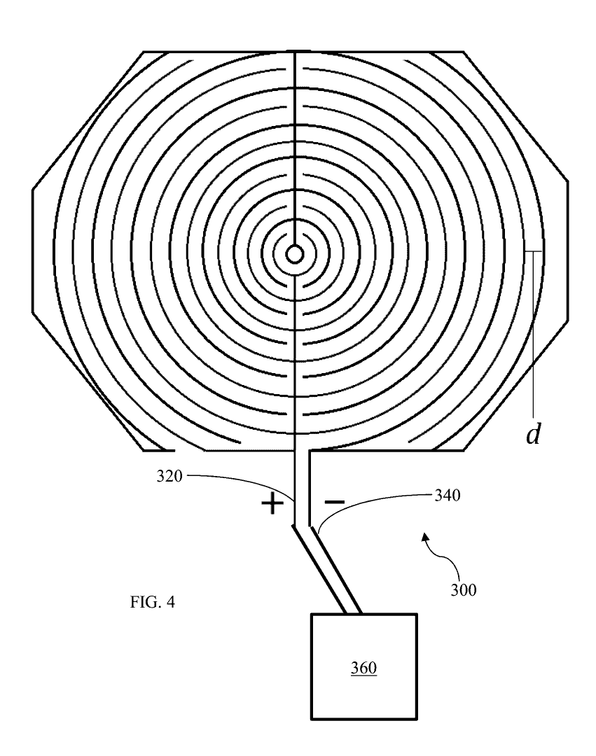

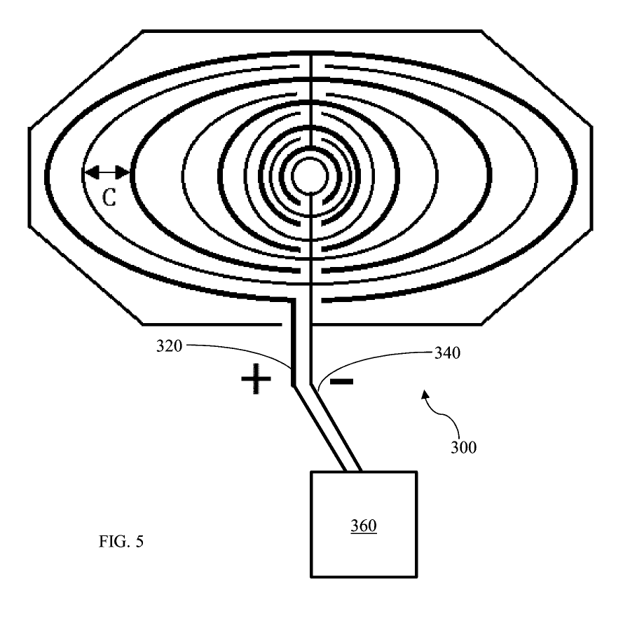

Various configurations of the electrodes 320, 340 are shown, for example in FIGS. 2, 4, and 5. The electrodes 320, 340 are preferably arranged to be in communication with the measuring device 360 (i.e., an appropriate analog or digital measuring device, or a computer device), and are configured to cooperate therewith, so as to allow the measuring device 360 to measure the change in an electrical property of the pressure-sensitive element 200, in response to the pressure-sensitive element 200 experiencing the change in pressure applied thereto, the change in deflection thereof, or the strain experienced thereby. In one example, the electrodes 320, 340 may be comprised of a conductive material, such as copper, a conductive fabric, etc., deposited on the surface of the film or pressure-sensitive element 200. In some aspects, the electrodes 320, 340, may be disposed between the pressure-sensitive element 200, and an additional pressure-sensitive element (not shown). That is, the electrodes 320, 340 may be sandwiched between a pair of pressure-sensitive elements, as necessary or desired, for the purposes as disclosed herein. In such instances, the electrodes 320, 340 may be configured to cooperating with the measuring device 360, so as to allow the measuring device 360 to measure the change in the electrical property of either or both of the pressure-sensitive elements, in response to either or both of the pressure-sensitive elements experiencing the change in pressure applied thereto, the change in deflection thereof, or the change in strain experienced thereby.

In particular aspects, the electrodes 320, 340 are comprised of a conductive material (i.e., copper) deposited on the one or more pressure-sensitive elements in any suitable pattern such that the positive and negative electrodes 320, 340 are separated by a dimension (i.e., length, width, or thickness) of the pressure-sensitive element 200. As such, any pressure applied to the pressure-sensitive element 200, any deflection or bending of the pressure-sensitive element 200, or any strain experienced by the pressure-sensitive element 200 may, in turn, change the resistivity or conductivity of the pressure-sensitive element 200 or, for example, a change in the voltage drop between the positive and negative electrodes 320, 340, which can be determined by the measuring arrangement 400 using conventional techniques of measuring electrical parameters. Aspects of the disclosed pressure-sensitive element 200, with certain properties (i.e., resistivity or conductivity) which actively change in response to stimuli in the form of pressure and/or deflection, may thus be monitored by way of the engaged electrodes 320, 340 to provide on-demand or constant feedback on the state of the flexible film and thus the surface (the eye/eyelid/orbital region) to which the film is applied. The pressure-sensitive element 200 may thus provide a responsive and reliable indication of inadvertent external pressure or contact acting on the eye/eyelid/orbital region.

In some instances, particularly in a surgical procedure where the patient is required to be in a prone position, the patient's facial area may be supported by a headrest device, which necessarily contacts the patient's face to support the patient's head. If the patient's head is not properly positioned in the headrest, or the patient's head otherwise shifts in the headrest during the surgical procedure, the headrest may become a source of extraocular pressure on the eye/orbital region of the patient which, in turn, may cause central retinal artery occlusion (CRAO), and thus post-operative vision loss. However, relatively small, and typically visually imperceptible, changes in the shape or geometry of the eye/orbit area may be caused by some likely indicators of ischemia resulting in vision loss, namely increased venous congestion, intraorbital pressure, or intracranial pressure resulting from extended surgeries in the prone position, and which may contribute to or cause post-operative vision loss. Some optical-related diseases, such as glaucoma, may also cause pressure within the eye itself to become elevated, wherein the elevated pressure may fluctuate under various conditions. As such, aspects of the present disclosure, by way of the apparatus 100 implementing the pressure-sensitive element 200 and associated measuring arrangement 300 may allow the eye/orbital region of the patient to be closely monitored during a surgical procedure, without undue action required on the part of the surgical team, and may also provide an indication of any change in pressure affecting the eye/orbital region, as such changes may occur, such that the surgical team may take timely remedial action to relieve the pressure on the patient's eye/orbital region, before permanent post-operative vision loss can occur.

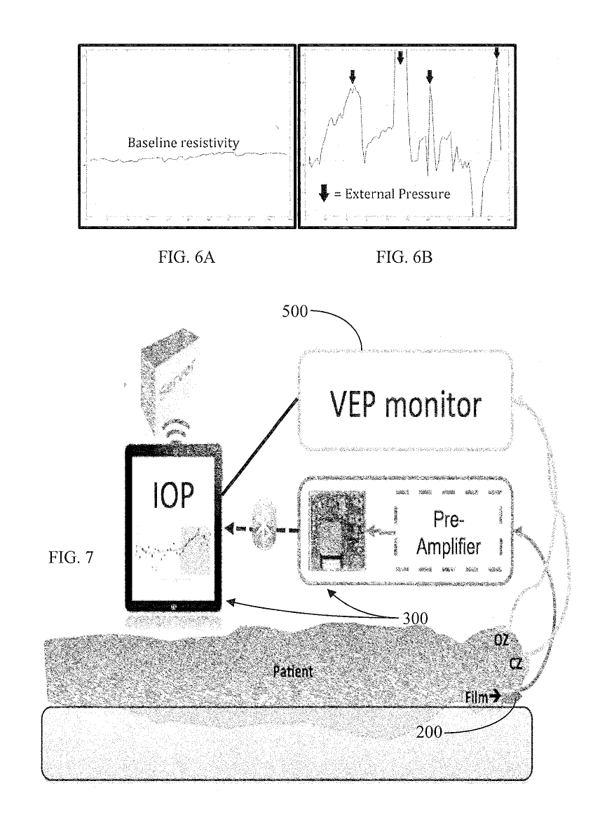

In one aspect involving monitoring of pressure affecting the eye/orbital region, the measuring arrangement 300 may be configured to determine a baseline of the property (i.e., resistivity or conductivity) of the pressure-sensitive element 200 applied to the orbital area, prior to the pressure-sensitive element 200 experiencing the change in pressure applied thereto. That is, for example, upon application of the pressure-sensitive element 200 to the orbital region of the anesthetized patient (i.e., before the patient is placed in the prone position with the patient's head supported by the headrest), the measuring arrangement 300 (i.e., the measuring device 360) may be actuated to measure a property of the pressure-sensitive element 200 (i.e., resistivity or conductivity), for instance, either by a single measurement or according to the average of a number of measurements over a time period. This initial analysis thus serves to form the baseline resistivity/conductivity of the orbital region of the patient prior to the onset of the surgical procedure, and before the patient is placed in the prone position in preparation for the surgical procedure (see, e.g., FIG. 6A). Deviations of the monitored pressure (see, e.g., FIG. 6B) from this baseline may thereafter be indicative of changes in pressure and/or deflection from the initial condition of the eye/orbital area upon application of the pressure-sensitive element 200. In some instances, such a deviation may also indicate separation of the pressure-sensitive element from the orbital region, which may be timely corrected prior to surgery from the skin. As such, the measuring arrangement 300 (i.e., the measuring device 360) may be configured to provide a visual indicium or an auditory indicium, if the pressure applied to the orbital area exceeds a threshold deviation from the baseline, as determined by the apparatus 100. That is, because the property (i.e., resistivity or conductivity) of the pressure-sensitive element 200 can be continually monitored by the measuring arrangement 300 (i.e., the measuring device 360), and since pressure affecting the eye/orbital area may vary under certain conditions (but not to the level of causing post-operative vision loss), the measuring arrangement 300 may be configured to provide an alert, whether visual or auditory or both, should the measuring arrangement 300 determine a pressure, or a change in pressure, affecting the eye/orbital region which exceeds a selected threshold and which may cause post-operative vision loss in the patient, if the pressure is not relieved in a timely manner.

In some aspects, other measures for monitoring the eye/orbital area of the patient may also be implemented in conjunction with the disclosed apparatus 100. For example, in some instances, the pressure-sensitive element 200 may include a light source 400 (i.e., a light-emitting diode or LED) engaged therewith, wherein the light source 400 may be engaged with the pressure-sensitive element 200 so as to be disposed over the eye when the pressure-sensitive element 200 is applied to the orbital area. In doing so, the light source 400 may be configured to emit light (i.e., in the form of a momentary emission or “flash”) toward the eye for eliciting a visual evoked potential response in an occipital cortex of the patient or for conducting an electroretinography procedure on the patient, in order to assess continued visual function. In some instances, the assessment of continued visual function in this manner may allow the collected response data to be correlated, for example, with the length of the surgical procedure (i.e., any change in visual function over the duration of the procedure), the state of the eye/orbital area (i.e., in relation to any changes in pressure which may be determined via the pressure-sensitive element 200), patient demographic data, and/or minute-to-minute physiologic data (i.e., EKG, pulse oximetry, arterial pressure), either by the measuring arrangement 300 or through interfacing with the measuring device 360. That is, in some aspects as shown, for example, in FIG. 7, the measuring arrangement 300/measuring device 360 may be interfaced with an electrophysiology device or an electroretinography system (see, e.g., element 500) for correlation of data associated therewith (i.e., visually-evoked potential (VEP) amplitude and latency changes, or electroretinography results) with the determined change in the property (i.e., resistivity or conductivity) of the pressure-sensitive element 200. In other instances, the determined change in the property (i.e., resistivity or conductivity) of the pressure-sensitive element 200 may be correlated with patient intraoperative physiologic data, including blood pressure, heart rate, and depth of anesthesia.

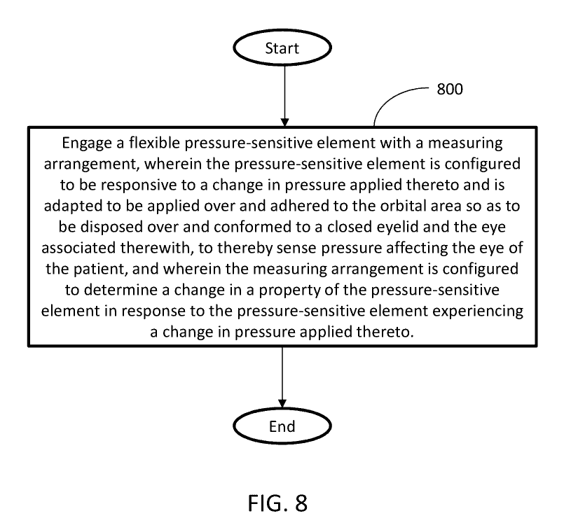

Another aspect of the disclosure herein is directed to a method of forming an apparatus for monitoring an orbital area, including an eye, of a patient during surgery, as shown, for example, in FIG. 8. Such a method comprises engaging a flexible pressure-sensitive element with a measuring arrangement, wherein the pressure-sensitive element is configured to be responsive to a change in pressure applied thereto and is adapted to be applied over and adhered to the orbital area so as to be disposed over and conformed to a closed eyelid and the eye associated therewith, to thereby sense pressure affecting the eye of the patient, and wherein the measuring arrangement is configured to determine a change in a property of the pressure-sensitive element in response to the pressure-sensitive element experiencing a change in pressure applied thereto (block 800).

In relation to such a method of manufacture, a temporary adhesive material may be engaged with the pressure-sensitive element, wherein the temporary adhesive material cooperates with the pressure-sensitive element to adhere the pressure-sensitive element to the orbital area to hold the eyelid closed. In some instances, the pressure-sensitive element may be engaged with a substrate having a temporary adhesive material applied thereto, wherein the substrate is configured to cooperate with the temporary adhesive material to adhere the substrate to the orbital area to hold the eyelid closed, and such that the pressure-sensitive element is disposed over the eye of the patient.

Aspects of such a method may further comprise applying electrodes to the pressure-sensitive element such that the electrodes are spaced apart by a selected dimension of the pressure-sensitive element, and such that the electrodes are in communication with the measuring arrangement and are capable of cooperating therewith to measure the change in an electrical property of the pressure-sensitive element, in response to the pressure-sensitive element experiencing the change in pressure applied thereto. In some instances, the electrodes may be applied between the pressure-sensitive element, and an additional pressure-sensitive element, such that the electrodes are capable of cooperating with the measuring arrangement to measure the change in the electrical property of either or both of the pressure-sensitive elements, in response to either or both of the pressure-sensitive elements experiencing the change in pressure applied thereto.

The measuring arrangement may include a measuring device, with such aspects of the method comprising engaging the measuring device into electrical communication with the electrodes, such that the measuring device is capable of cooperating with the electrodes to determine a change in resistivity or conductivity of the pressure-sensitive element, or a change in a voltage drop between the electrodes, in response to deflection of the pressure-sensitive element, pressure applied to the pressure-sensitive element, or strain experienced by the pressure-sensitive element.

Aspects of the method may also include forming the pressure-sensitive element by distributing conductive elements within a nonconductive matrix, such that the matrix having the conductive elements dispersed therein is configured to change resistivity or conductivity in response to deflection thereof, pressure applied thereto, or strain experienced thereby. The matrix may be comprised of a polymeric material, such as polyethylene, while the conductive elements distributed therein may comprise of carbon black, carbon nanotubes, graphite, a metal, or a metal oxide, within the nonconductive matrix.

A light source may be engaged with the pressure-sensitive element, such that the light source is disposed over the eye when the pressure-sensitive element is applied to the orbital area, so that the light source is capable of emitting light toward the eye for eliciting a visual evoked potential response in an occipital cortex of the patient or for conducting an electroretinography procedure on the patient. In addition, the measuring arrangement may be interfaced with an electrophysiology device or an electroretinography system for correlation with the determined change in the property of the pressure-sensitive element.

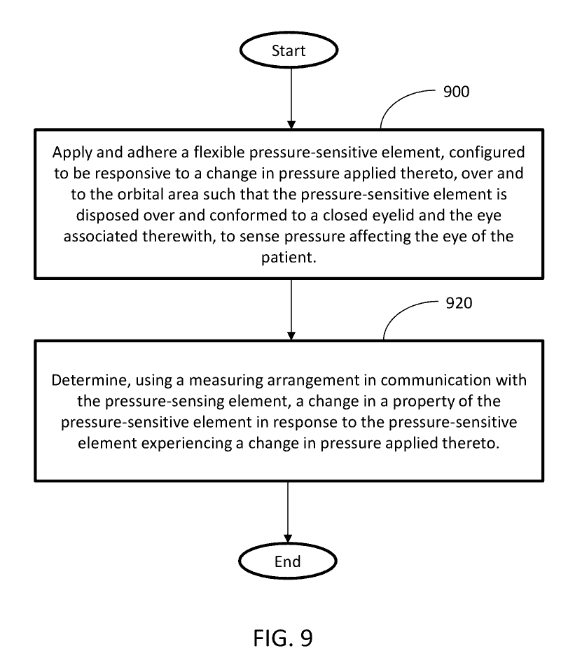

Another aspect of the disclosure herein is directed to a method of monitoring an orbital area, including an eye, of a patient during surgery, as shown, for example, in FIG. 9. Such a method comprises applying and adhering a flexible pressure-sensitive element, configured to be responsive to a change in pressure applied thereto, over and to the orbital area such that the pressure-sensitive element is disposed over and conformed to a closed eyelid and the eye associated therewith, to sense pressure affecting the eye of the patient (block 900). Using a measuring arrangement in communication with the pressure-sensing element, a change in a property of the pressure-sensitive element in response to the pressure-sensitive element experiencing a change in pressure applied thereto, is then determined (block 920).

Aspects of such a method may further comprise adhering the pressure-sensitive element over and to the orbital area using a temporary adhesive material engaged with the pressure-sensitive element so as to hold the eyelid closed. In some instances, the pressure-sensitive element may be engaged with a substrate having a temporary adhesive material applied thereto, and the substrate having the pressure-sensitive element engaged therewith then adhered over and to the orbital area to hold the eyelid closed, and such that the pressure-sensitive element is disposed over the eye of the patient.

Such a method may further comprise applying electrodes to the pressure-sensitive element such that the electrodes are spaced apart by a selected dimension of the pressure-sensitive element. With the electrodes in communication with the measuring arrangement, the method may comprise measuring the change in an electrical property of the pressure-sensitive element, in response to the pressure-sensitive element experiencing the change in pressure applied thereto. In some instances, the electrodes may be applied between the pressure-sensitive element, and an additional pressure-sensitive element. With the electrodes in communication with the measuring arrangement, the method may comprise measuring the change in the electrical property of either or both of the pressure-sensitive elements, in response to either or both of the pressure-sensitive elements experiencing the change in pressure applied thereto.

In some aspects, the measuring arrangement may include a measuring device engaged in electrical communication with the electrodes, and the method may comprise determining, with the measuring device, a change in resistivity or conductivity of the pressure-sensitive element, or a change in a voltage drop between the electrodes, in response to deflection of the pressure-sensitive element, pressure applied to the pressure-sensitive element, or strain experienced by the pressure-sensitive element.

The pressure-sensitive element may be formed by distributing conductive elements within a nonconductive matrix, such that the matrix having the conductive elements dispersed therein is configured to change resistivity or conductivity in response to deflection thereof, pressure applied thereto, or strain experienced thereby.

In some aspects, light may be emitted toward the eye (i.e., as a momentary emission or flash from a light-emitting diode or LED), using a light source engaged with the pressure-sensitive element and disposed over the eye when the pressure-sensitive element is applied to the orbital area, for eliciting a visual evoked potential response in an occipital cortex of the patient or for conducting an electroretinography procedure on the patient.

In some instances, a baseline of the property of the pressure-sensitive element applied to the orbital area may first be determined using the measuring arrangement, prior to the pressure-sensitive element experiencing the change in pressure applied thereto, wherein a deviation of the property from the baseline is indicative of pressure applied to the orbital area. If the pressure applied to the orbital area exceeds a threshold deviation from the baseline, the method may comprise providing a visual indicium or an auditory indicium using the measuring arrangement. Further, the measuring arrangement may be interfaced with an electrophysiology device or an electroretinography system for correlation with the determined change in the property of the pressure-sensitive element, and/or the measuring arrangement may be used to correlate the determined change in the property of the pressure-sensitive element with patient intraoperative physiologic data, including blood pressure, heart rate, and depth of anesthesia.

Many modifications and other aspects of the disclosure will come to mind to one skilled in the art to which this disclosure pertains having the benefit of the teachings presented in the foregoing descriptions and the associated drawings. For example, rapid eye movement (REM) sleep may involve rapid and random movement of the eye. In such instances, the movement of the eye may, in turn, cause movement or deflection of the eyelid which, further in turn, may cause the pressure-sensitive element 200 of the type disclosed herein to experience a change in the pressure exerted thereon, a change in the deflection thereof, or a change in the strain experienced thereby. As such, aspects of the present disclosure may be applicable, for example to the detection and analysis of REM sleep. Further, one skilled in the art will appreciate that, though exemplary aspects of the present disclosure are directed to pressure experienced by the eye, other aspects of the present disclosure may also be applied to the determination of orbital (extraocular) pressure changes and associated pressure effects associated therewith (i.e., due to an orbital pseudotumor, due to thyroid eye disease, or relative to intraocular pressure), by appropriately adjusting the configuration of the pressure-sensitive element 200 and/or the measuring arrangement 300 in communication therewith to differentiate the anatomy for which the pressure changes and effects thereof are being determined. Therefore, it is to be understood that the disclosure is not to be limited to the specific aspects disclosed herein and that modifications and other aspects are intended to be included within the scope of the appended claims. Although specific terms are employed herein, they are used in a generic and descriptive sense only and not for purposes of limitation.

That which is claimed:

1. An apparatus for monitoring an orbital area, including an eye, of a patient during surgery, the apparatus comprising:
a flexible pressure-sensitive element configured to be responsive to a change in pressure applied thereto, the pressure-sensitive element being adapted to be applied over and adhered to the orbital area so as to be disposed over and conformed to a closed eyelid and the eye associated therewith, to sense pressure affecting the eye of the patient; and
a measuring arrangement in communication with the pressure-sensing element, the measuring arrangement being configured to determine a change in a property of the pressure-sensitive element in response to the pressure-sensitive element experiencing a change in pressure applied thereto.

2. The apparatus of claim 1, comprising a temporary adhesive material engaged with the pressure-sensitive element and cooperating therewith to adhere the pressure-sensitive element to the orbital area to hold the eyelid closed.

3. The apparatus of claim 1, comprising a substrate having a temporary adhesive material applied thereto, and having the pressure-sensitive element engaged therewith, the substrate cooperating with the temporary adhesive material to adhere the substrate to the orbital area to hold the eyelid closed, and such that the pressure-sensitive element is disposed over the eye of the patient.

4. The apparatus of claim 1, comprising electrodes applied to the pressure-sensitive element and spaced apart by a selected dimension of the pressure-sensitive element, the electrodes being in communication with the measuring arrangement and cooperating therewith to measure the change in an electrical property of the pressure-sensitive element, in response to the pressure-sensitive element experiencing the change in pressure applied thereto.

5. The apparatus of claim 4, wherein the electrodes are disposed between the pressure-sensitive element, and an additional pressure-sensitive element, the electrodes cooperating with the measuring arrangement to measure the change in the electrical property of either or both of the pressure-sensitive elements, in response to either or both of the pressure-sensitive elements experiencing the change in pressure applied thereto.

6. The apparatus of claim 4, wherein the measuring arrangement includes a measuring device engaged in electrical communication with the electrodes, the measuring device being configured to cooperate with the electrodes to determine a change in resistivity or conductivity of the pressure-sensitive element, or a change in a voltage drop between the electrodes, in response to deflection of the pressure-sensitive element, pressure applied to the pressure-sensitive element, or strain experienced by the pressure-sensitive element.

7. The apparatus of claim 1, wherein the pressure-sensitive element comprises a nonconductive matrix having conductive elements distributed therein, and wherein the matrix having the conductive elements dispersed therein is configured to change resistivity or conductivity in response to deflection thereof, pressure applied thereto, or strain experienced thereby.

8. The apparatus of claim 7, wherein the matrix is comprised of a polymeric material.

9. The apparatus of claim 8, wherein the matrix is comprised of polyethylene.

10. The apparatus of claim 7, wherein the conductive elements are comprised of carbon black, carbon nanotubes, graphite, a metal, or a metal oxide.

11. The apparatus of claim 1, comprising a light source engaged with the pressure-sensitive element, the light source being disposed over the eye when the pressure-sensitive element is applied to the orbital area so as to emit light toward the eye for eliciting a visual evoked potential response in an occipital cortex of the patient or for conducting an electroretinography procedure on the patient.

12. The apparatus of claim 11, wherein the light source comprises a light emitting diode configured for momentary emission of the light directed to the eye.

13. The apparatus of claim 1, wherein the measuring arrangement is configured to determine a baseline of the property of the pressure-sensitive element applied to the orbital area prior to the pressure-sensitive element experiencing the change in pressure applied thereto, and wherein a deviation of the property from the baseline is indicative of pressure applied to the orbital area.

14. The apparatus of claim 13, wherein the measuring arrangement is configured to provide a visual indicium or an auditory indicium, if the pressure applied to the orbital area exceeds a threshold deviation from the baseline.

15. The apparatus of claim 1, wherein the measuring arrangement is configured to interface with an electrophysiology device or an electroretinography system for correlation with the determined change in the property of the pressure-sensitive element.

16. The apparatus of claim 1, wherein the measuring arrangement is configured to correlate the determined change in the property of the pressure-sensitive element with patient intraoperative physiologic data, including blood pressure, heart rate, and depth of anesthesia.

17. A method of forming an apparatus for monitoring an orbital area, including an eye, of a patient during surgery, the method comprising:
engaging a flexible pressure-sensitive element with a measuring arrangement, the pressure-sensitive element being configured to be responsive to a change in pressure applied thereto and being adapted to be applied over and adhered to the orbital area so as to be disposed over and conformed to a closed eyelid and the eye associated therewith, to thereby sense pressure affecting the eye of the patient, the measuring arrangement being configured to determine a change in a property of the pressure-sensitive element in response to the pressure-sensitive element experiencing a change in pressure applied thereto.

18. The method of claim 17, comprising engaging a temporary adhesive material with the pressure-sensitive element, the temporary adhesive material cooperating with the pressure-sensitive element to adhere the pressure-sensitive element to the orbital area to hold the eyelid closed.

19. The method of claim 1, comprising engaging the pressure-sensitive element with a substrate having a temporary adhesive material applied thereto, the substrate being configured to cooperate with the temporary adhesive material to adhere the substrate to the orbital area to hold the eyelid closed, and such that the pressure-sensitive element is disposed over the eye of the patient.

20. The method of claim 1, comprising applying electrodes to the pressure-sensitive element such that the electrodes are spaced apart by a selected dimension of the pressure-sensitive element, and such that the electrodes are in communication with the measuring arrangement and are capable of cooperating therewith to measure the change in an electrical property of the pressure-sensitive element, in response to the pressure-sensitive element experiencing the change in pressure applied thereto.

21. The method of claim 20, wherein applying electrodes comprises applying the between the pressure-sensitive element, and an additional pressure-sensitive element, such that the electrodes are capable of cooperating with the measuring arrangement to measure the change in the electrical property of either or both of the pressure-sensitive elements, in response to either or both of the pressure-sensitive elements experiencing the change in pressure applied thereto.

22. The method of claim 20, wherein the measuring arrangement includes a measuring device, and the method comprises engaging the measuring device into electrical communication with the electrodes, such that the measuring device is capable of cooperating with the electrodes to determine a change in resistivity or conductivity of the pressure-sensitive element, or a change in a voltage drop between the electrodes, in response to deflection of the pressure-sensitive element, pressure applied to the pressure-sensitive element, or strain experienced by the pressure-sensitive element.

23. The method of claim 17, comprising forming the pressure-sensitive element by distributing conductive elements within a nonconductive matrix, such that the matrix having the conductive elements dispersed therein is configured to change resistivity or conductivity in response to deflection thereof, pressure applied thereto, or strain experienced thereby.

24. The method of claim 23 wherein forming the pressure-sensitive element comprises forming the pressure-sensitive element by distributing the conductive elements within a matrix comprised of a polymeric material.

25. The method of claim 24 wherein forming the pressure-sensitive element comprises forming the pressure-sensitive element by distributing the conductive elements within a matrix comprised of polyethylene.

26. The method of claim 23 wherein forming the pressure-sensitive element comprises forming the pressure-sensitive element by distributing conductive elements comprised of carbon black, carbon nanotubes, graphite, a metal, or a metal oxide, within the nonconductive matrix.

27. The method of claim 17, comprising engaging a light source with the pressure-sensitive element, such that the light source is disposed over the eye when the pressure-sensitive element is applied to the orbital area, so that the light source is capable of emitting light toward the eye for eliciting a visual evoked potential response in an occipital cortex of the patient or for conducting an electroretinography procedure on the patient.

28. The method of claim 17, comprising interfacing the measuring arrangement with an electrophysiology device or an electroretinography system for correlation with the determined change in the property of the pressure-sensitive element.

29. A method of monitoring an orbital area, including an eye, of a patient during surgery, the method comprising:
applying and adhering a flexible pressure-sensitive element, configured to be responsive to a change in pressure applied thereto, over and to the orbital area such that the pressure-sensitive element is disposed over and conformed to a closed eyelid and the eye associated therewith, to sense pressure affecting the eye of the patient; and
determining, using a measuring arrangement in communication with the pressure-sensing element, a change in a property of the pressure-sensitive element in response to the pressure-sensitive element experiencing a change in pressure applied thereto.

30. The method of claim 29, wherein adhering the pressure-sensitive element comprises adhering the pressure-sensitive element over and to the orbital area using a temporary adhesive material engaged with the pressure-sensitive element so as to hold the eyelid closed.

31. The method of claim 29, comprising engaging the pressure-sensitive element with a substrate having a temporary adhesive material applied thereto, and wherein adhering the pressure-sensitive element comprises adhering the substrate having the pressure-sensitive element engaged therewith over and to the orbital area to hold the eyelid closed, and such that the pressure-sensitive element is disposed over the eye of the patient.

32. The method of claim 29, comprising applying electrodes to the pressure-sensitive element such that the electrodes are spaced apart by a selected dimension of the pressure-sensitive element.

33. The method of claim 32, wherein the electrodes are in communication with the measuring arrangement, and the method comprises measuring the change in an electrical property of the pressure-sensitive element, in response to the pressure-sensitive element experiencing the change in pressure applied thereto.

34. The method of claim 32, wherein applying the electrodes comprises applying the electrodes between the pressure-sensitive element, and an additional pressure-sensitive element.

35. The method of claim 34, wherein the electrodes are in communication with the measuring arrangement, and the method comprises measuring the change in the electrical property of either or both of the pressure-sensitive elements, in response to either or both of the pressure-sensitive elements experiencing the change in pressure applied thereto.

36. The method of claim 32, wherein the measuring arrangement includes a measuring device engaged in electrical communication with the electrodes, and wherein the method comprises determining, with the measuring device, a change in resistivity or conductivity of the pressure-sensitive element, or a change in a voltage drop between the electrodes, in response to deflection of the pressure-sensitive element, pressure applied to the pressure-sensitive element, or strain experienced by the pressure-sensitive element.

37. The method of claim 29, comprising forming the pressure-sensitive element by distributing conductive elements within a nonconductive matrix, such that the matrix having the conductive elements dispersed therein is configured to change resistivity or conductivity in response to deflection thereof, pressure applied thereto, or strain experienced thereby.

38. The method of claim 29, comprising emitting light toward the eye, using a light source engaged with the pressure-sensitive element and being disposed over the eye when the pressure-sensitive element is applied to the orbital area, for eliciting a visual evoked potential response in an occipital cortex of the patient or for conducting an electroretinography procedure on the patient.

39. The method of claim 38, wherein emitting light toward the eye comprises emitting light toward the eye using a light emitting diode configured for momentary emission of the light.

40. The method of claim 29, comprising determining a baseline of the property of the pressure-sensitive element applied to the orbital area using the measuring arrangement, prior to the pressure-sensitive element experiencing the change in pressure applied thereto, and wherein a deviation of the property from the baseline is indicative of pressure applied to the orbital area.

41. The method of claim 40, comprising providing a visual indicium or an auditory indicium using the measuring arrangement, if the pressure applied to the orbital area exceeds a threshold deviation from the baseline.

42. The method of claim 29, comprising interfacing the measuring arrangement with an electrophysiology device or an electroretinography system for correlation with the determined change in the property of the pressure-sensitive element.

43. The method of claim 29, comprising correlating, using the measuring arrangement, the determined change in the property of the pressure-sensitive element with patient intraoperative physiologic data, including blood pressure, heart rate, and depth of anesthesia.
